(19) United States
(12) Patent Application Publication
(10) Pub. No.: US 2007/0253943 A1
(43) Pub. Date: Nov. 1, 2007

(54) Compositions for Topical Treatment

(76) Inventor: Ali Altunkaya, Adana (TR)

Correspondence Address: BROOKS KUSHMAN P.C., 1000 TOWN CENTER, TWENTY-SECOND FLOOR, SOUTHFIELD, MI, 48075, US

(21) Appl. No.: 11/570,806

(22) PCT Filed: Jun. 16, 2005
(86) PCT No.: PCT/IB05/01692

§ 371 Date: Dec. 18, 2006

(30) Foreign Application Priority Data
Jun. 22, 2004 (GB) 0413954.9

(51) Int. Cl. A61K 38/44; A61P 17/00; A61P 17/06

(52) U.S. Cl. 424/94.4

(57) ABSTRACT

Compositions are disclosed for topical treatment of mammalian skin, particularly human skin, relieve the distressing symptoms of the many skin diseases and provide, in many cases, a long term cure. The composition comprise, in combination, one or more boswellic acids and/or a fatty acid having a chain length of at least 18 carbon atoms and which contains at least two unsaturated linkages together with an enzyme based antibacterial system. The compositions will selectively reduce cell turnover and may be used for the treatment of eczema and/or psoriasis.

This invention relates to compositions for topical treatment of mammalian skin and particularly human skin.

Persistent skin diseases exist which are extremely resistant to remedial treatment. In particular eczema and psoriasis, among other skin diseases such as impetigo, Pemphigus Vulgaris, Roscicea folliculitis are responsible for considerable patient discomfort and disfigurement. Remedial treatments involving diet and topical application of medication provide relief in a few cases but little long term improvement.

It is principle advantage of the present invention to provide compositions for treatment of psoriasis, eczema and other undesirable skin disorders which are of relatively simple and inexpensive design and operation. The compositions are free from undesirable side effects, effectively treat the symptoms and can provide a cure for undesirable skin diseases.

A further advantage of the present invention is the provision of a method for treatment of psoriasis or other undesirable skin disorder which reduces and/or eliminates “itchiness” of skin which is a common symptom/condition of such skin disorders.

The present invention provides compositions for topical application to skin surfaces which relieve the distressing symptoms of the above afflictions and provide, in may cases, a long term cure.

According to the present invention there is provided a composition for topical application to mammalian skin comprising, in combination, one or more boswellic acids and an enzyme based antibacterial system.

There is also provided a composition for topical application to mammalian skin comprising, in combination, a fatty acid having a chain length of at least 18 carbon atoms and which contains at least two unsaturated linkages and an enzyme based antibacterial system.

There is further provided a composition for topical application to mammalian skin comprising, in combination, one or more boswellic acids, a fatty acid having a chain length of at least 18 carbon atoms and which contains at least two unsaturated linkages and an enzyme based antibacterial system.

The enzyme based antibacterial system is preferably a mixture of one or more enzymes, buffers and substrates that convert oxygen into oxidative compounds having antibacterial action. The antibacterial agent selectively inhibits high growth keratinocyte cells yet not affecting normal growth of cells. The main enzymes are preferably lactoperoxidase and glucose oxidase, with glucose present as a substrate and the mixture is buffered to a pH in the range 5 to 6.5, most preferably in the range 5.3 to 5.6. The enzyme based antibacterial system is preferably activated by admixture with a combination of potassium iodide and sodium thiocyanate.

The fatty acid or combination of fatty acids are either saturated or unsaturated and containing 8 to 28 carbon atoms. Preferably the composition is in the form of a cream emulsion for topical application containing from 5 to 40% by weight of caprylic capric triglycerides, sunflower, safflower, soybean, corn, evening primrose, refined herring or tuna oil and other oil as carrier. It may also contain from 2 to 20% by weight of glycerine, propylene glycol or other humectant with a moisturising property. The lipid components of the composition preferably form a discontinuous phase in an aqueous emulsion containing and in which water comprises fro 15 to 85% by weight of the emulsion.

The compositions according to the invention will selectively reduce cell turnover and reduce inflammation of the skin. Such compositions can cure or alleviate symptoms of psoriasis. They will also cure or alleviate symptoms of psoriatic arthritis and arthritis.

While the invention may be embodied in many different forms, specific preferred embodiments of the invention are now described. The method for treatment of psoriasis or other skin disorders such as dry skin, eczema, itchy skin, red skin, itchy eczema, inflamed skin, and/or cracked skin involves the application of a topical ointment to the affected area of an individual's skin at least once per day and preferably twice per day for treatment of the undesirable skin condition.

A composition for the topical treatment of portions of skin of a person afflicted with psoriasis or other skin disorders such as dry skin, eczema, itchy skin, red skin, itchy eczema, inflamed skin, and/or cracked skin comprises four major components:
I selective anti-inflammatory agent(s) specific to eczema and psoriasis conditions to reduce inflammation and allow all other biological pathways to continue normally,
II antimicrobial agent(s) to prevent infection,
III anti-hyperproliferative cell agent(s) to slow down the excessive cell turn over, and
IV one or more compounds to restore the affected areas of skin to a normal condition.

All the components I to IV are contained in a pharmaceutically acceptance carrier. The compositions may be blended with other components to form a liquid, gel or an emulsion in cream form to assist in application to the skin and retention after application.

The preferred anti-inflammatory agents (I) occur in nature and are based on:
A Boswellia serrata extract, which contains boswellic acids, is a known natural anti-inflammatory agent which selectively inhibits the formation of leuketrines (pro-inflammatory agents produced by the body).
B Polyunsaturated fatty acids or their esters, also known as essential fatty acids, with a minimum carbon chain length of 18 e.g. lipoleic acid and/or its esters.

The boswellic acids (A) are naturally occurring substances derived from the resin of the plant Boswellia serrata and other Boswellia species such as Boswellia carteri. The resin is available under the name frankincense or olibanuma and used for its aromatic properties. The boswellia acids found in the resin are based on a picene nucleus and occur in a number of isomeric forms. The naturally occurring mixtures may include components with nuclear substituents. The mixed acids are commercially available. Methods of separating different components of the naturally occurring Boswellic acids are described in EP-A-0 755 940. It has been found that boswellic acids act as lipoxygenase inhibitors.

The fatty acids (B) have two unsaturated linkages and may be pure compounds or in the form of a commercial oil, such as sunflower, soya, rapeseed, borage, and other vegetable oils or fish oils such as tuna or herring.

Fatty acids having a chain length of greater than 18 carbon atoms are well known. Many occur naturally in a free state or more commonly as triglycerides. The esterified acids are easily released by hydrolysis. The fatty acids occur in many isomeric forms, straight chains, branched chains, and may contain one or more unsaturated linkages. Such fatty acids are commercially available.

The preferred anti-bacterial systems are sold under the trade marks “Myavert-C” and “Biovert”. These systems are a mixture of natural enzymes, buffers and substrates that convert oxygen, which may be atmospheric, into oxidative compounds having anti-bacterial action. The main enzymes used are lactoperioxidase and glucose oxidase. Glucose is present as a substrate and the mixture is buffered to a pH in the range 5.0 to 6.5, preferably 5.3 to 5.6 The system is activated by admixing a combination of potassium iodide and sodium thiocyanate. The enzymes oxidase the iodide ions to iodate and the thiocyanate ions to hypothiocyanite. The oxidised ions selectively oxidise bacteria and fungal cell walls killing both bacteria and fungi.

The antibacterial action of the combination of these ingredients is described in WO-A-91/11105. Other enzyme based anti-bacterial systems, such as lactoferrin, are also effective in the control of bacteria and fungi in the compositions of the present invention.

It has been discovered that the combination of boswellic acids with the enzyme systems and also the anti-inflammatory agents based on polyunsaturated fatty acids and/or their esters e.g. linoleic acid or ethyl linolate with the compounds of the anti microbial agent e.g. glucose oxidase and lactoperoxidase, as used in the compositions of this invention, selectively slow down the cell turn over of SVK-14 cells (immortalised keratinocyte cell lines derived from human neonatal foreskin that are hyperproliferative) but has no effect on normal cells. This action overcomes psoriasis.

Well known moisturisers, emollients and humectants can be used as skin restorer. The preferred compounds are based on pure linoleic acid or plant derived oils that are rich in linoleic acid.

The compositions of this invention in one preferred form contain the anti-inflammatory agent, linoleic acid, and the enzyme based anti-microbial system in a pharmaceutically acceptable carrier.

Due to its unsaturated nature linoleic acid (pure or as part of plant oil) is not stable when exposed to air and is easily degraded, mainly by oxidation, to aldehyde and alcohol derivatives. To prevent such degradation the majority of commercially available plant oils and purified linoleic acid contain tocopherol (Vitamin E) as an antioxidant. The presence of tocopherol in the compositions of the invention has a detrimental effect on the ability of linoleic acid to slow down the cell turnover of keratinocyte cells. The linoleic acid used in the compositions of the invention should be substantially free from any antioxidants.

The enzyme based anti-microbial system (Myavert C) functions initially by reacting with the available oxygen to form peroxides which in turn are consumed by the lactoperoxidase to produce active species that destroy bacteria and fungi. The presence of the enzyme of the compositions of the invention appears to stablilise the linoleic acid since it removes oxygen and hence prevents the oxidation of linoleic acid.

The linoleic acid in the compositions of this invention performs five functions:
1. it acts as an anti-inflammatory agent;
2. it reduces selectively the cell turn over of hyperproliferative cells;
3. it softens the hard skin present;
4. it acts as a moisturiser and an emollient and
5. it helps to restore the stratum corneum to normal so that it can perform its barrier function (prevent water loss, ingress of chemicals and micro organisms).

The overall function of the enzyme based antibacterial system “Myavert C” in the composition of this invention is:
1. to remove oxygen from the enclosed system (e.g. a cream in a tube or jar) and hence stabilise the linoleic acid;
2. to act as a preservative to stabilise the product;
3. to destroy anti-microbials from the skin surface in use and
4. to reduce selectively the cell turn over of hyperproliferative cells.

The topical compositions of this invention are provided for use in a pharmaceutically acceptable carrier and this could take the form of a solution, cream, ointment, emulsion or balm or any other form well known in the art and the preferred carrier form is an oil in water emulsion cream.

The Table below lists the preferred components of the compositions/carriers according to this invention. Alternative carriers well known in the art and based on GRAS (generally regarded as safe) components could be substituted for the base given below.

Function and Range of Components

| Component | Function | Range, % w/w |
|---|---|---|
| water | carrier phase | 20-80 |
| Permulen TR2 NF | emulsifier | up to 2 |
| (Carbomer) | | |
| Lactic Acid | pH buffer | up to 5 |
| Carbopol 980NF | thickener | up to 2 |
| Glycerine | humectant | up to 20 |
| Caprylic/capric | Pharmaceutical | up to 80 |
| triglycerides | solvent, solubiliser | |
| | for Boswellia | |
| Boswellia | Anti-inflammatory | up to 25 |
| Linoleic Acid | | up to 25 |
| Sunflower oil | emollient | up to 25 |
| Polyoxyethylene | surfactant | up to 10 |
| sorbitan monooleate | | |
| 2,4-dichlorobenzyl | antifungal agent | up to 2 |
| alcohol | | |
| Sodium lactate | pH buffer | up to 5 |
| Myavert C | Preservative and | up tp 25 |
| | anti-bacterial agent | |

Formulations

The following Table gives examples of formulations.

| | | | | | 15B6 | | | | |
|---|---|---|---|---|---|---|---|---|---|
| Ingredients | 6A | 6B | 44 | 15A6 | % w/w | 18A | 18B | 20A | 20B |
| Water I | 13.60 | 17.60 | | 13.20 | 13.20 | 24.55 | 28.55 | 5.15 | 5.88 |
| Lactic Acid | 0.25 | 0.25 | 0.25 | 0.25 | 0.25 | — | — | 0.20 | 0.24 |
| (90%) | | | | | | | | | |
| Citric Acid | — | — | — | — | — | 0.15 | 0.15 | — | — |
| Pemulen TR2 NF | 13.70 | 13.70 | 13.70 | 15.00 | 15.00 | 13.70 | 13.70 | 15.00 | 17.66 |
| (3.0% solution) | | | | | | | | | |
| Carbopol 900 NF | 20.40 | 20.40 | 20.40 | 17.50 | 17.50 | 20.40 | 20.40 | 15.00 | 17.66 |
| (2.0% solution) | | | | | | | | | |
| Glycerine | 5.00 | 5.00 | 5.00 | 5.00 | 5.00 | 4.00 | 4.00 | 4.00 | 4.71 |
| Caprylic Capric | 36.00 | 36.00 | 36.00 | 24.00 | 24.00 | 16.00 | 16.00 | 25.00 | 29.43 |
| Triglycarides | | | | | | | | | |
| Roswellia serata | 4.00 | | 4.00 | — | — | 4.00 | — | 15.00 | — |
| with total | | | | | | | | | |
| boswellic acid | | | | | | | | | |
| isomers | | | | | | | | | |
| Sunflower oil | — | — | — | — | 16.50 | 12.00 | 12.00 | 12.00 | 14.12 |
| Linoleic Acid- | — | — | 20.00 | 16.50 | — | — | — | — | — |
| 92.6% active | | | | | | | | | |
| level (with - | | | | | | | | | |
| 50 ppm nat vit E) | | | | | | | | | |
| Surfacare T80 | 0.30 | 0.30 | 0.30 | 0.30 | 0.30 | 0.25 | 0.25 | 0.40 | 0.47 |
| (Polyoxycthylene | | | | | | | | | |
| sorbitan | | | | | | | | | |
| monooleate) | | | | | | | | | |
| Myacide SP | 0.15 | 0.15 | 0.15 | 0.15 | 0.15 | 0.15 | 0.15 | 0.15 | 0.15 |
| (2,4-dichlorobenzyl] | 2.00 | 2.00 | | 2.00 | 2.00 | 2.00 | 2.00 | 2.00 | 2.35 |
| alcohol) Water | | | | | | | | | |
| II | | | | | | | | | |
| Sodium Lactate | 2.00 | 2.00 | 2.00 | 2.50 | 2.50 | | | 2.00 | 2.35 |
| (60%) | | | | | | | | | |
| Sodium citrate | — | — | — | — | — | 1.25 | 1.25 | — | — |
| TEA (50% sol.) | — | — | — | 1.00 | 1.00 | — | — | 1.50 | 2.30 |
| Myavert C part 1 | 2.00 | 2.00 | — | 2.00 | 2.00 | 1.00 | 1.00 | 2.00 | 2.00 |
| Water III | 0.50 | 0.50 | — | 0.50 | 0.50 | 0.50 | 0.50 | 0.50 | 0.59 |
| Myavert C part 2 | 0.10 | 0.10 | — | 0.10 | 0.10 | 0.05 | 0.05 | 0.10 | 0.10 |
| Total | 100.00 | 100.00 | 100.00 | 100.00 | 100.00 | 100.0 | 100.00 | 100.00 | 100.00 |

Typical Process of Preparation
The above formulations can be prepared using the Cold procedure
described below.

| Stage | Action |
|---|---|
| 1 | Prepare a 3.0% Solution of Premulen TR2 NF as |
| | described in manufactuer's procedure |
| 2 | Prepare a 2.0% Solution of Carobpol 980 NF as |
| | described in the manufacturer's procedure |
| 3 | Mix Boswellic Acid and Caprylic/Capric |
| | Triglycerides and heat to 115-120° C. Hold at this |
| | temperature for 30 minutes and then cool room |
| | temperature. The temperature of the mix must be |
| | maintained between 22-24° C. throughout this |
| | procedure4 |
| 4 | In a suitable mixing vessel with an anchor stirrer |
| | and homogeniser add Lactic Acid and water 1 and |
| | stir for 5 minutes. |
| 5 | Add Premulen TR2 NF solution from stage 1 and stir |
| | for 20 minutes. |
| 6 | Add Carbomer 908 NF solution from stage 2 and stir |
| | for 20 minutes. |
| 7 | Add glycerine, stir for 20 minutes. (WATER PHASE) |
| 8 | To Boswellia serrata and Caprylic/Capric |
| | Triglycerides solution of stage 3 at 22-24° C. add |
| | linoleic acid and/or sunflower oil, polyoxyethylene |
| | sorbitan monooleate and 2,4-dichlorobenzyl alcohol |
| | and stir for 30 minutes (OIL PHASE) |
| 9 | Ensure oil phase is about 2° C. above the temperature |
| | of water phase. |
| 10 | Add oil phase to water phase with rapid stirring |
| | over 5 minutes and continue mixing for 15 minutes |
| | to form an oil in water emulsion cream. |
| 11 | Cool the emulsion to contain temperature between |
| | 22-24° C. if necessary. |
| 12 | Dissolve sodium citrate in water 2 and add to the |
| | main emulsion mix with rapid stirring; stir for 5 |
| | minutes. |
| 13 | Check pH |
| 14 | Slow the stirrer to gentle mixing and adjust pH |
| | with TEA (50%) to 5.4-5.6 at and stir for 20 |
| | minutes. |
| 15 | Add Myavert C part 1 and stir for 15 minutes. |
| 16 | Dissolve Myavert C part 2 into water 3 and add to |
| | the emulsion and stir for 15 minutes. |
| 17 | Check and record final pH and viscosity of the |
| | emulsion (Specification pH = 5.4-5.6 and Viscosity = |
| | 20,0000-80,000 cps) |

Boswellia serrata containing compositions can also be prepared using the HOT procedure described below.

| Stage | Action |
|---|---|
| 1 | Prepare a 3.0% solution of Premulen TR2 NF as described |
| | in manufacturer's procedure |
| 2 | Prepare a 2.0% solution of Carbopol 980 NF as described |
| | in manufacturer's procedure |
| 3 | Mix Boswellic acids(s) and Caprylic/Capric Triglycerides |
| | and heat to 115-120° C. Hold at this temperature for 30 |
| | minutes and then cool 82-84° C. |
| 4 | In a suitable mixing vessel with an anchor stirrer and |
| | homogeniser add Lactic Acid and water 1 and stir for 5 |
| | minutes |
| 5 | Add Premulen TR2 NF solution from stage 1 and stir for 20 |
| | minutes |
| 6 | Add Carbomer 980 NF solution from stage 2 and stir for 20 |
| | minutes |
| 7 | Add glycerine, stir for 20 minutes then raise the |
| | temperature to 80-82° C. (WATER PHASE) |
| 8 | To Boswellic acid(s) and Caprylic/Capric Triglycerides |
| | solution of stage 3 at 82-84° C. add linoleic acid and/or |
| | sunflower oil, polyoxyethylene sorbitan monooleate and |
| | 2,4-dichlorobenzyl alcohol and stir for 30 minutes (OIL |
| | PHASE) |
| 9 | Ensure oil phase is about 2° C. above the temperature of |
| | water phase |
| 10 | Add oil phase to water phase with rapid stirring over 5 |
| | minutes and continue mixing for 15 minutes to form an oil |
| | in water emulsion cream |
| 11 | Start cooling the emulsion |
| 12 | Dissolve sodium citrate in water 2 and add to the main |
| | emulsion mix with rapid stirring at 45-50° C. V; stir for 5 |
| | minutes |
| 13 | Check pH |
| 14 | Slow the stirrer to gentle mixing and adjust pH with TEA |
| | (50%) to 5.4-5.6 at 40-45° C. and stir for 20 minutes |
| 15 | Add myavert to C part 1 and stir for 15 minutes |
| 16 | Dissolve Myavert C part 2 into water 3 and add to the |
| | emulsion and stir for 15 minutes |
| 17 | Check and record final pH and viscosity of the emulsion |
| | (Specification pH = 5.4-5.6 and Viscosity = 20,000-80,000 |
| | cps) |

Clinical Trials
Formulations 18A and 19B

A male child aged 2½ years (Patient No. 1/98) had atopic eczema covering more than 18% of the body surface. The eczema had been present for two weeks and left untreated. There was visual indication of bacterial infection. Eczema Cream 18A was applied to the affected areas of the patient on a regular basis. After 3 days the redness and irritation had subsided. After two months the pruritis was eliminated and all the symptoms of eczema were overcome within 2½ months, restoring the skin to its normal health. The patient remained in remission for at least three years. (This was the last point at which he was available for examination.)

22 patients were treated with cream 18A and 22 with cream 18B. Both creams overcame eczema on patients and 18A gave a faster response on average overcoming eczema in two weeks whereas 18B took 3 to 4 weeks. Patients treated with 18A showed a lower relapse rate than those treated with 18B.

Clinical Results of Pilot Study on the Effects of 15A and 15B Cream Formulations in the Treatment of Psoriasis

A clinical trial was carried out using selected creams (15A −15% of 94%=14.1%) active linoicic acid and enzyme based anti-bacterial system versus the (15B) enzyme based anti-bacterial system only in an emollient base, to establish the overall efficacy of the optimized formulations and their ability to control psoriasis.

Information was recorded in questionnaire, and where possible photographic, form. During the first consultation with patients one of two topical cream treatments, named either 15A or 15B, was prescribed with instructions to apply the cream twice a day up to two weeks after clearance of lesions. 15A is Linoleic acid (14.1%) and enzyme based anti-bacterial system in an emollient base. 15B is an enzyme based anti-bacterial system in an emollient base only. Progression in clinical state was recorded in questionnaire, note and photographic form, as and when the patients returned to the clinic.

Two male and four female participants were recruited from patients diagnosed with psoriasis and confirmed by biopsy attending a Dermatology Clinic. A wide range of participants (N) were recruited. Age and prior duration of the participants' psoriasis condition was recorded in years a percentage of the body area covered with psoriasis. The data was coded as; (A) 1% or less, (B) 1-5%, (C) 10%, (D) 10-18% and (E) greater than 18%. The ‘Types of Psoriasis’ were recorded as Plaque, Erythrodermic, Guttate (drop), Inverse, Pustular, Scalp, Nail, Psoriatic Arthritis, others to be specified) at first visit and or following biopsy. A ‘Body map’ was marked by circling the effected area and ‘Site of Lesion’, was specified and coded as, (0) OK, (1) Mild, (2) Moderate, (3) Moderate to Severe, and (4) Severe. Lesions were also recorded in photographic form where the patient consented. The presence of fungal infection to visual examination was noted as either ‘Yes’, ‘No’ or ‘Do not know’, The ‘Site of lesion’ coding was also used to score the characteristics ‘Exfoliation/Scalines’ ‘Pain, ‘Redness and irritation’, ‘Pruritus’, and ‘Pustules’, Detailed demographic data collected for each of the participants. The measure of response to treatment was recorded as the number of days taken for all lesions on all parts of the body to clear named, ‘Cleared’. Improvement, clearance, and remission data is presented in the Table below.

Improvement and Clearance For Participants
Treated with 15A and 15B

| | | | Remission after | | | Last |
|---|---|---|---|---|---|---|
| Partici- | Improvement | Cleared | clearance | | Total | recorded |
| pant | seen at | Nos of | Nos of | Nos. of | Nos. of | visit Nos. |
| Number | Day | days | days | visits | visits | of days |

PARTICIPANTS TREATED WITH 15A

| | | | | | | |
|---|---|---|---|---|---|---|
| 1 | 21, 35, 63, | 147 | 51 | 2 | 9 | 198 |
| | 91, 119 | | | | | |
| 3 | 4 and 72 | 72 | — | — | 3 | 72 |
| 4 | 7, 14, and | 44 | — | — | 4 | 44 |
| | 44 | | | | | |
| 5 | 14 | 30 | — | — | 3 | 30 |
| 6 | 24 | 56 | — | — | 3 | 47 |

PARTICIPANTS TREATED WITH 15B

| | | | | | | |
|---|---|---|---|---|---|---|
| 2 | 24 and 40 | 40 | — | — | 3 | 40 |

1. A composition for topical application to mammalian skin comprising, in combination, one or more boswellic acids and an enzyme based antibacterial system.

2. A composition for topical application to mammalian skin comprising, in combination, a fatty acid having a chain length of at least 18 carbon atoms and which contains at least two unsaturated linkages and an enzyme based antibacterial system comprising at least two enzymes.

3. A composition according to claim 2 wherein the enzyme based antibacterial system comprises lactoperoxidase and glucose oxidase.

4. A composition according to claim 3 wherein the lactoperoxidase and glucose oxidase are present in an amount of from 0.05 to 0.10% by weight, based on the weight of the composition.

5. A composition for topical application to mammalian skin comprising, in combination, one or more boswellic acids, an enzyme based antibacterial system and a fatty acid having a chain length of at least 18 carbon atoms and which contains at least two unsaturated linkages.

6. A composition for topical application comprising an enzyme based antibacterial system in an emollient base.

7. A composition claimed in claim 1, in which the enzyme based antibacterial system is a mixture of at least two enzymes, buffers and substrates that convert oxygen into oxidative compounds having antibacterial action.

8. A composition as claimed in claim 7, in which the antibacterial agent selectively inhibits high growth keratinocyte cells yet not affecting normal growth of cells.

9. A composition as claimed in claim 7, in which the main enzymes are lactoperoxidase and glucose oxidase, with glucose present as a substrate and the mixture is buffered to a pH in the range 5 to 6.5.

10. A composition as claimed in claim 9, in which the mixture is buffered to a pH in the range 5.3 to 5.6.

11. A composition as claimed in claim 1, in which the enzyme based antibacterial system is activated by admixture with a combination of potassium iodide and sodium thiocyanate.

12. A composition as claimed in claim 1 in which a fatty acid or a combination of fatty acids either saturated or unsaturated and containing 8 to 28 carbon atoms is present as a delivery agent.

13. A composition as claimed in claim 1 in the form of a cream emulsion for topical application containing from 5 to 40% by weight of sunflower, safflower, soyabean, corn, evening primrose, refined herring or tuna oil and any other oil as carrier.

14. A composition as claimed in claim 1, containing from 10 to 80% by weight of caprylic/capric triglycerides.

15. A composition as claimed in claim 1, containing from 2 to 20% by weight of glycerine, propylene glycol and any other humectant with a moisturizing property.

16. A composition as claimed in claim 10, in which the lipid components form a discontinuous phase in an aqueous emulsion in which water comprises from 15 to 85% by weight of the emulsion.

17. A composition as claimed in claim 1 that will selectively reduce cell turnover.

18. The use of a composition as claimed in claim 1 in therapy.

19. The use of a composition as claimed in claim 1 in the manufacture of a medicament for the treatment of eczema and psoriasis.

20. The use of a composition as claimed in claim 2 in the manufacture of a medicament for the treatment of psoriasis.

21. The use of a composition as claimed in claim 5 in the manufacture of a medicament for the combined treatment of psoriasis and eczema.

22. Method for making compositions as claimed in claim 1 as herein described.

23. A method for the treatment of psoriasis comprising administering to a patient in need thereof an effective amount of a composition according to claim 1.

24. A method for the treatment of eczema comprising administering to a patient in need thereof an effective amount of a composition according to claim 1.
